(19) United States
(12) Patent Application Publication
(10) Pub. No.: US 2026/0199691 A1
(43) Pub. Date: Jul. 16, 2026

(54) ANCHORING ARRANGEMENT FOR ANCHORING AN IMPLANTABLE MEDICAL DEVICE AT TISSUE OF A PATIENT

(71) Applicant: BIOTRONIK SE & Co. KG, Berlin (DE)

(72) Inventors: Devan HUGHES, Tualatin, OR (US); Eric AUSTIN, Portland, OR (US); Matthew MELIUS, Milwauki, OR (US); Valerie RODRIGUEZ, Banks, OR (US); Thomas KUSKE, Rostock (DE); Michael STIEHM, Rostock (DE); Stefan SIEWERT, Rostock (DE); Klaus-Peter SCHMITZ, Rostock (DE)

(73) Assignee: BIOTRONIK SE & Co. KG, Berlin (DE)

(21) Appl. No.: 19/136,644

(22) PCT Filed: Dec. 5, 2023
(86) PCT No.: PCT/EP2023/084332

§ 371 Date: Jun. 6, 2025

Related U.S. Application Data
(60) Provisional application No. 63/431,445, filed on Dec. 9, 2022.

(30) Foreign Application Priority Data
Feb. 14, 2023 (EP) 23156558.1

(51) Int. Cl. A61N 1/375

(52) U.S. Cl. CPC A61N 1/37518; A61N 1/37512; A61N 1/3756

(57) ABSTRACT

An anchoring arrangement for anchoring an implantable medical device at tissue of a patient includes a fixation member for fixing the anchoring arrangement to the implantable medical device and at least two tines extending from the fixation member at opposite sides relative to a central axis of the anchoring arrangement. The tines have a curved geometry and are elastically deformable such as to be reversibly deformable between a relaxed configuration and a pre-deployment configuration. The curved geometry and deformation characteristics of the tines are configured such that, in an initial phase of a deformation procedure during which the tines deform from their pre-deployment configuration to their relaxed configuration due to their intrinsic prestress, the cantilever ends of the tines are displaced such as to follow a deployment trajectory which extends at an emergence attack angle of between 40° and 90° relative to the central axis of the anchoring arrangement.

### CROSS-REFERENCE TO RELATED APPLICATIONS

This application is the United States National Phase under 35 U.S.C. §371 of PCT International Patent Application No. PCT/EP2023/084332, filed on Dec. 5, 2023, which claims the benefit of European Patent Application No. 23156558.1, filed on Feb. 14, 2023, and U.S. Provisional Patent Application No. 63/431,445 , filed on Dec. 9, 2022, the disclosures of which are hereby incorporated by reference herein in their entireties.

### TECHNICAL FIELD

The present invention relates to an anchoring arrangement for anchoring an implantable medical device in the tissue of a patient. Furthermore, the present invention relates to an implantable medical device comprising such anchoring arrangement.

### BACKGROUND

There are various medical devices (also referred to as implantable medical devices—IMD) which must be implanted into a patient at locations inside the patient's body. Specifically, there are medical devices which may be active, i.e. which may for example generate electrical signals and transmit such signals to tissue inside the patient's body and which are therefore referred to as active IMDs (AIMD). For example, pacemakers may be implanted into a patient to generate electrical signals for stimulating the patient's heart.

Conventionally, pacemakers comprised a housing and an elongate lead extending from the housing, an end of the lead serving as an electrode. The housing was to be implanted subcutaneously. The electrode was then implanted to directly electrically contact cardiac tissue. However, leads for AIMDs have been found to be a potential source of clinical complications.

In order to avoid such clinical complications, implantable leadless pacemakers (ILP, sometimes also referred to as intracardiac pacemakers) have been developed. Leadless pacemakers are miniaturized devices which are configured to be implanted directly into a patient's heart, therefore requiring no external lead. Typically, an ILP is implanted directly into a heart chamber such as one of the ventricles. The ILP comprises a housing accommodating electronics such as a controller and further accommodating an energy source such as a battery. Furthermore, the ILP comprises an electrode which is exposed at an outside of the housing. Finally, the ILP comprises an anchoring arrangement which is configured to anchor the housing to cardiac tissue enclosing e.g. the ventricle such as to reliably fix the ILP inside the heart chamber with the ILP's electrode being in electrical contact to the cardiac tissue.

Generally, during an implantation procedure, an ILP is introduced into the patient's body, advanced to an intended implantation location in the patient's heart and fixed at such intended location using a specific anchoring arrangement. For delivering the ILP to the intended location in the heart, the ILP may be attached to or comprised in a distal end of a specific delivery system. For example, during a delivery procedure, the ILP may be accommodated within a protector sheath (sometimes also referred to as protector cup). The delivery system typically comprises an elongate catheter. Therein, the catheter may be steerable and/or guidable such that a distal end of the catheter may be displaced throughout the patient's body, i.e. for example along vessels of the patient, until reaching the intended implantation location. Upon having reached the implantation location, the ILP may be deployed, i.e. may be ejected from the protector sheath. During a deployment procedure, the anchoring arrangement may be activated such as to anchor the ILP in tissue at the implantation location.

For example, the anchoring arrangement may comprise a plurality of elastically deflectable tines. When being arranged in the protector sheath, these tines may be deformed in a pre-deployment configuration in which the tines are mechanically deformed and intrinsically prestressed into a geometry in which they may be comprised within the typically tubular protector sheath. In the deployment procedure, the ILP together with its anchoring arrangement may be successively ejected from the protector sheath such that the tines leave the protector sheath at a distal end thereof. As the protector sheath was held against the cardiac tissue at the implantation site, the tines may then penetrate and engage the cardiac tissue. Upon resuming their relaxed, non-stressed configuration, the tines may then reliably anchor the ILP to the cardiac tissue.

There may be a need for an improved anchoring arrangement for anchoring an implantable medical device at tissue of a patient. Particularly, there may be a need for an anchoring arrangement enabling anchoring an IMD at various types of cardiac tissue, also including cardiac tissue differing from the cardiac tissue enclosing the heart's ventricle and having different characteristics in comparison to such ventricular cardiac tissue. Specifically, there may be a need for an anchoring arrangement enabling anchoring an IMD, in particular an ILP, in cardiac tissue enclosing a heart's atrium. Furthermore, there may be a need for an IMD comprising such anchoring arrangement.

The present disclosure is directed toward overcoming one or more of the above-mentioned problems, though not necessarily limited to embodiments that do.

### SUMMARY

Such needs may be met with the subject-matter of the independent claims. Advantageous embodiments are defined in the dependent claims as well as in the corresponding specification and figures.

According to a first aspect of the present invention, an anchoring arrangement configured for anchoring an implantable medical device, in particular an implantable leadless pacemaker, in the tissue of a patient, in particular at the atrium of a patient's heart, is described. The anchoring arrangement comprises a fixation member for fixing the anchoring arrangement to the implantable medical device and at least two tines extending from the fixation member at different sides relative to a central axis of the anchoring arrangement. The tines have a curved geometry and are elastically deformable such as to be reversibly deformable between a relaxed configuration and a pre-deployment configuration. In the relaxed configuration, the tines are free of any intrinsic mechanical prestress and middle portions of the tines are arranged distally relative to the fixation member and cantilever ends of the tines are directed proximally and/or arranged proximally relative to the fixation member. In the pre-deployment configuration, the tines are mechanically deformed and intrinsically prestressed into a geometry in which the anchoring arrangement is arrangeable in a tubular protector sheath with the middle portions as well as the cantilever ends of the tines being arranged and directed distally relative to the fixation member. Therein, the curved geometry and deformation characteristics of the tines are configured such that, in an initial phase of a deformation procedure during which the tines deform from their pre-deployment configuration to their relaxed configuration due to their intrinsic prestress, the cantilever ends of the tines are displaced such as to follow a deployment trajectory which extends at an emergence attack angle of between 40° and 90° relative to the central axis of the anchoring arrangement.

The phrase “at least two tines extending from the fixation member at different sides relative to a central axis of the anchoring arrangement” is to be understood within the frame of the application as tines distributed along the circumference of a circle having the center at the central axis of the anchoring arrangement. If the angle of at least two of the tines and the center of the circle is at least 100°, in particular at least 120°, in particular at least 180°, the two tines are considered at different sides relative to a central axis of the anchoring arrangement.

According to a second aspect of the invention, an implantable medical device, in particular an implantable leadless pacemaker in particular configured for implantation within the atrium of a patient's heart, is described. The IMD comprises a housing, a controller comprised in the housing for controlling functions of the medical device, an energy source comprised in the housing for supplying the controller and an anchoring arrangement according to an embodiment of the first aspect of the invention with its fixation member being fixed to the housing.

Ideas underlying embodiments of the present invention may be interpreted as being based, inter alia, on the following observations and recognitions.

Briefly summarised in a non-limiting manner, embodiments of the present invention relate to an anchoring arrangement by which an IMD such as an ILP may be fixed at implantation locations which differ from conventionally used implantation locations with regards to, inter-alia, characteristics of the tissue to which the IMD is to be fixed and/or characteristics of an environment and space in which the IMD is to be arranged.

Particularly, it has been found that, for specific medical conditions, it may be beneficial to implant an ILP inside an atrial chamber instead of implanting the ILP in a ventricular chamber. However, characteristics of cardiac tissue enclosing an atrium significantly differ from those of cardiac tissue enclosing a ventricle for example with respect to a thickness of the tissue, surface characteristics as well as elasticity characteristics of the tissue and a geometry of the tissue. Specifically, atrial cardiac tissue is generally much thinner, softer and/or more compliant than ventricular cardiac tissue and, furthermore, typically comprises no smooth surface but comprises, at least in partial areas, a pectinate structure.

It was therefore an objective to design e.g. an anchoring arrangement serving as a fixation mechanism that secures the IMD, in particular an ILP, within an atrial chamber of the heart while also achieving reliable atrial electrode to tissue contact. The fixation should not damage the atrium unnecessarily or to a point of clinical dysfunction or cause unacceptable loss of blood.

In order to fulfil such objectives, it has been found that the tines of the anchoring arrangement have to be specifically configured. For example, while tines of a conventional anchoring arrangement for anchoring an ILP to ventricular tissue are typically configured for relatively deeply penetrating the ventricular tissue, the tines of the anchoring arrangement proposed herein shall be specifically configured for only shallow tissue penetration.

For such purpose, the geometry and elastic deformation characteristics of the tines are specifically adapted such that, when the IMD with its anchoring arrangement is deployed from a tubular protector sheath during an implantation and deployment procedure, the tines extend at an angle (referred to herein as emergence attack angle) of between 40° and 90° relative to the central axis of the anchoring arrangement. In other words, as the central axis of the anchoring arrangement is generally orthogonal to a surface of the cardiac tissue at which the anchoring arrangement is to be fixed, the tines shall extend at a shallow angle of less than 50°, preferably less than 40°, less than 30°, less than 20°, less than 10° or even at about 0° (i.e. in parallel) relative to the surface of the cardiac tissue and therefore penetrate such cardiac tissue and/or, optionally, pectinate structures at such cardiac tissue at a shallow angle. Upon penetrating the cardiac tissue at such shallow angle, the cantilever ends of the tines may then be displaced along a shallow deployment trajectory.

Accordingly, as a result of the deployment procedure, the tines may extend through the cardiac tissue with a small penetration depth, thereby significantly reducing the risk of damaging or even perforating the cardiac wall enclosing e.g. an atrial chamber. Furthermore, the geometry of the tines may be configured such as to take account of the restricted available space within an atrial chamber.

In the following, characteristics of embodiments of the present invention will be described in more detail.

The anchoring arrangement described herein is specifically adapted for anchoring an IMD, in particular an ILP, in the tissue of a patient, particularly in cardiac tissue enclosing an atrial chamber of the patient's heart. The anchoring arrangement comprises a fixation member to be fixed at the IMD with tines for penetrating the tissue in order to anchor the IMD at the cardiac tissue.

The fixation member may form a common base element or root element from which the tines extend. The fixation member may consist of a single element. For example, the fixation member may comprise a ring, plate or other structural component and further including attachment structures via which the fixation member may be fixed to the IMD. The fixation member may have a symmetrical shape including for example a mirror symmetry or a rotational symmetry relative to a symmetry axis. For example, the fixation member may have a circular shape.

The tines extend laterally from the fixation member. Preferably, the tines extend in a radially outward direction from the fixation member. Accordingly, a root end of each tine is attached to the fixation member whereas an opposite end is unsupported and is therefore referred to as a cantilever end. Preferably the tines may be distributed equidistant from each other along the circumference of a circle having the center at the central axis of the anchoring arrangement. In an embodiment, the anchoring arrangement comprises an even number of tines circumferentially equidistant from each other, i.e. e.g. two, four, six, eight or more tines. In this embodiment, the tines are arranged in pairs wherein tines of a pair extend in diametrically opposite directions and thereby at opposite sides relative to the central axis of the anchoring arrangement. Alternatively, the anchoring arrangement comprises an odd number of tines circumferentially equidistant from each other, i.e. three, five, seven or more tines. In this embodiment at least two of the tines are arranged at different sides relative to a central axis of the anchoring arrangement. The tines extend from the fixation member at opposite sides relative to the central axis of the anchoring arrangement, wherein such central axis may coincide with a longitudinal middle axis and/or a symmetry axis of the fixation member and/or of the IMD. The tines may extend from the fixation member in an equidistant configuration and/or in a symmetric configuration relative to e.g. the central axis. Preferably, but not necessarily, the tines are unitary with the fixation member, i.e. the tines and the fixation member form a single component with the tines integrally extending from the fixation member. The tines may consist of a medical-grade material. Furthermore, the tines may be made with a material having a shape memory effect. Preferably, the tines may consist of a super-elastic material, particularly of a super-elastic metal or metal alloy such as a nickel-titanium alloy, also referred to as Nitinol. For example, the tines may be fabricated by laser cutting from a Nitinol tube and setting to a desired shape, such setting for example including forming the tines into an intended curved geometry and further including a thermal treatment for establishing a shape memory effect. The tines may have a consistent or homogeneous thickness and/or width along their entire length. Alternatively, the tines may have a heterogeneous thickness and/or width along their entire length. At the cantilever end, each tine tip may end in a radius. All tines of the anchoring arrangement may have a same length. Alternatively, the anchoring arrangement may comprise tines of different lengths wherein, for example, tines of a first type of tines are longer than tines of a second type of tines. Preferably, there are at least two tines of each type of tines. Furthermore, there may be an even number of tines of each type of tines. The tines or, optionally, the entire anchoring arrangement may be subjected to finishing processes such as bead blasting, etching and/or electro-polishing to remove sharp edges and/or reduce a propensity for corrosion.

The tines are fabricated to have a curved geometry when being in their relaxed configuration. Therein, the relaxed configuration corresponds to a configuration in which no substantial external forces are applied to the tines and no intrinsic mechanical prestress is present in the tines. Accordingly, the relaxed configuration corresponds to a shape which the tines acquire due to their super-elastic and shape memory effect, i.e. when the tines are not actively deformed. As described in further details below, the curved geometry of the tines may be specifically adapted such that the tines may penetrate tissue during the deployment procedure in an intended manner, particularly with an intended emergence attack angle and enabling shallow tissue penetration. Particularly, in the relaxed configuration, each tine comprises a middle portion which is arranged distally relative to the fixation member, i.e. the middle portion of the tine is arranged further away from the IMD than the fixation member. Furthermore, in the relaxed configuration, each tine has its cantilever end arranged such that the cantilever end directs proximally, i.e. in a proximal direction which extends towards the IMD, and/or each tine has its cantilever end arranged proximally relative to the fixation member.

Due to their super-elasticity, the tines may be temporarily and reversibly deformed into other configurations such as a pre-deployment configuration. In such pre-deployment configuration, the anchoring arrangement together with the IMD may be accommodated within a tubular protector sheath. Generally, the diameter of such protector sheath is significantly smaller than the diameter of the anchoring arrangement in its relaxed configuration such that the tines of the anchoring arrangement have to be substantially deformed in their pre-deployment configuration. Particularly, in the pre-deployment configuration, the middle portion is as well as the cantilever end of the tines are arranged and directed distally relative to the fixation member. Furthermore, in the pre-deployment configuration, the tines are deflected in a distal direction and towards the central axis and are therefore elastically prestressed, i.e. mechanically biased, such that an intrinsic force pushes the tines in a radially outward direction and/or proximal direction towards the relaxed configuration.

Therein, the curved geometry of the tines as well as the deformation characteristics of the tines are specifically adapted such that, when the tines are ejected from the tubular protector sheath, they begin to move and change their configuration in order to finally reassume their relaxed configuration. In such deformation procedure, the cantilever ends of the tines are displaced along a deployment trajectory upon the tines deflecting from the pre-deployment configuration to the relaxed configuration. Specifically, the curved geometry and deformation characteristics of the tines are adapted such that, in an initial phase of the deformation procedure, i.e. at the beginning of the deployment trajectory, the cantilever end of the tines move at an emergence attack angle relative to the central axis of the anchoring arrangement, this emergence attack angle being between 40° and 90°. The lower limit for the emergence attack angle may be larger than 40°, for example 45°, 50°, 55°, 60°, 65°, 70° or more. The upper limit for the emergence attack angle may be smaller than 90°, for example 89°, 88°, 87°, 85°, 82° or less. Accordingly, during deployment of the anchoring arrangement including the deformation procedure of the tines, the cantilever ends of the tines impinge onto and penetrate the surface of the adjacent tissue under a shallow penetration angle of between 0° and 50°. Therein, the penetration angle is defined to be complementary to the emergence attack angle, i.e. (penetration angle)=90°−(emergence attack angle). Generally, the smaller the penetration angle, the less the tines engage into the tissue, i.e. the smaller is the maximum penetration depth of the tines, and, as a result, the lower is a risk of damaging or even perforating a thin wall layer of tissue with the tines.

Preferably, according to an embodiment, the curved geometry and deformation characteristics of the tines are configured such as to implement an emergence attack angle of between 75° and 90°. More specifically, a lower limit for the emergence attack angle may be larger than 75°, for example 76°, 77°, 78°, 80°, 82°, 84° or more.

According to an embodiment, the curved geometry and deformation characteristics of the tines are configured such that a maximum penetration depth of the tines is less than 1 mm. Therein, the maximum penetration depth corresponds to a maximum distance, measured in a direction parallel to the central axis of the anchoring arrangement, of the deployment trajectory in relation to a position of the cantilever ends of the tines upon the tines being in their pre-deployed configuration.

The maximum penetration depth may also be interpreted as a distance by which the tines are introduced into the tissue in a direction parallel to the central axis and distal to the distal-most surface of the IMD. Expressed differently, the maximum penetration depth may be interpreted as a layer thickness of a layer engaged behind by the penetrated tines.

Preferably, the maximum penetration depth is limited to less than 0.95 mm, less than 0.9 mm, less than 0.85 mm, less than 0.8 mm, less than 0.75 mm, less than 0.7 mm, less than 0.65 mm, less than 0.6 mm, less than 0.55 mm, less than 0.5 mm, less than 0.45 mm, less than 0.4 mm, less than 0.35 mm, less than 0.3 mm, less than 0.25 mm, less than 0.2 mm, less than 0.15 mm or less than 0.1 mm. The deployment trajectory may even be adapted such that the maximum penetration depth is substantially 0 mm which means that the tines are ejected from the protector sheath at an emergence attack angle of 90° such as to extend substantially parallel to the surface of the tissue at which the tines are to be engaged. While, with such penetration angle of substantially 0°, the tines may not penetrate the tissue in a depth direction, the tines nevertheless may penetrate protrusions of the tissue formed by e.g. pectinate structures, as they are typically present at the surface of cardiac tissue at the atrium wall. Generally, the smaller the maximum penetration depth is set, the lower a risk of damaging or perforating a thin wall layer of tissue with the tines may be.

According to an embodiment, the curved geometry and deformation characteristics of the tines are configured such that an active maximum diameter of the anchoring arrangement is less than 15 mm. Therein, the active maximum diameter corresponds to a maximum width, measured in a direction orthogonal to the central axis of the anchoring arrangement, of the deployment trajectory.

Generally the active maximum diameter may correspond to or, in most cases, may be slightly larger than the diameter of the anchoring arrangement in its relaxed configuration. The active maximum diameter depends on the trajectory along which the cantilever ends of the tines travel during the deployment procedure, such trajectory potentially extending along a wider width than the width of the anchoring arrangement in its relaxed configuration at the end of the deployment procedure.

Preferably, the upper limit for the active maximum diameter is set to be less than 14.5 mm, less than 14 mm, less than 13.5 mm or less than 13 mm. However, the active maximum diameter should generally be substantially larger than the diameter of the IMD, such IMD typically having a diameter of between 5 mm and 12 mm, more typically between 6 mm and 9 mm. Accordingly, the lower limit for the active maximum diameter should generally be larger than 8 mm, in most cases larger than 10 mm or even larger than 12 mm.

With the anchoring arrangement having tines being configured for establishing a relatively small active maximum diameter, a risk of damaging or perforating a cardiac wall may be lowered when for example implanting the IMD with its anchoring arrangement in an atrium chamber with very restricted available space or volume. Furthermore, the small active maximum diameter may also increase a likelihood of the tines returning to their preset shape when engaged with tissue. If the active maximum diameter is too large, the small anatomy for example in an atrium chamber may restrict a tine movement such that the tines may not return to their preset relaxed shape.

According to an embodiment, each tine comprises, in the relaxed configuration, a first curved portion close to the fixation member and a second curved portion further distant from the fixation member.

In other words, each tine may comprise two curved portions arranged behind each other along the longitudinal extension of the tine. The two curved portions may differ from each other with respect to at least one characteristics including a radius of curvature, a direction of the curvature, a variation with which the curvature changes along the curved portion, a length of the curved portion, a sweep angle over which the curved portion extends, a width of the tine in the curved portion, a cross-section of the tine in the curved portion, deformation characteristics of the tine along the curved portion, etc.

By suitably adjusting or optimising characteristics of the at least two curved portions, deformation characteristics of the anchoring arrangement upon being deformed into the pre-deployment configuration may be influenced such that the tines, during a deformation procedure in a deployment process, travel along an intended deployment trajectory, particularly at an intended emergence attack angle and/or with an intended maximum penetration depth.

According to an embodiment, the first curved portion has a first radius of curvature and the second curved portion has a second radius of curvature different from the first radius of curvature.

For example, the first radius of curvature may be larger than the second radius of curvature. This may lower a mechanical strain in the first curved portion of the tine when being deformed into the pre-deployment configuration, i.e. when retained in the protector sheath. Furthermore, a spring force at the second curved portion of the tine may be strengthened. Alternatively, the first radius of curvature may be smaller than the second radius of curvature. Generally, the first radius of curvature may differ from the second radius of curvature by more than 2%, more than 5%, more than 10% or even more than 20%. In case the radius of curvature varies along a curved portion, the term “radius of curvature” may relate to an average radius of curvature along the curved portion.

According to an embodiment, the second curved portion encloses a sweep angle of between 80° and 150°.

The sweep angle may be interpreted as an angle between a first direction, in which the tine extends at a distal end of the second curved portion, and a second direction, in which the tine extends at a proximal end of the second curved portion. A lower limit for such sweep angle may be more than 85°, more than 90°, more than 95°, more than 100°, more than 105°, more than 110°, more than 115°, more than 120°, more than 125° or more than 130°. An upper limit for the sweep angle may be less than 145°, less than 140°, less than 135°, less than 130°, less than 125°, less than 120°, less than 115°, less than 110°, less than 105°, less than 100°or less than 95°.

Generally, the sweep angle at the second curved portion of the tine may influence a manner in which the cantilever end of the tine travels upon the deployment process, i.e. influence the deployment trajectory. By suitably setting the sweep angle at the second curved portion and, optionally, optimising such sweep angle in dependence of other characteristics of the tine such as a curvature, a length, deformation characteristics, etc. of adjacent portions of the tine, the deployment trajectory may be set such as to realise an intended emergence attack angle and/or maximum penetration depth.

According to an embodiment, each tine may further comprise, in the relaxed configuration, a proximal straight portion in between the first curved portion and the second curved portion. Additionally or alternatively, each tine may further comprise, in the relaxed configuration, a distal straight portion in between the second curved portion and the cantilever end.

In other words, the first curved portion and the second curved portion of the tine may be separated from each other by an intermediate straight portion referred to herein as proximal straight portion. Furthermore or as an alternative, a straight portion referred to herein as distal straight portion may be present between the second curved portion and the distal end of the respective tine. Characteristics of the proximal straight portion and/or of the distal straight portion including, inter-alia, a length of the respective straight portion may significantly influence the deformation characteristics of the tines. For example, the length of the proximal straight portion and/or of the distal straight portion may be set to be longer, shorter or equal to the length of the first curved portion and/or the length of the second curved portion. Furthermore, the length of the proximal straight portion may be larger, smaller or equal to the length of the distal straight portion. Accordingly, by suitably configuring such characteristics of the straight portion(s), the deployment trajectory may be set such as to realise an intended emergence attack angle and/or maximum penetration depth. The distal straight portion may be oriented in parallel to the central axis of the anchoring arrangement and in particular the distal straight portion may be shorter than the proximal straight portion.

Each tine comprises a tine width and a tine thickness, whereby the tine width is at least twice the tine thickness. The tine width and the tine thickness are perpendicular to the longitudinal direction of the tine. In an embodiment at least one tine may comprise a first curved portion having a tine width tapering from a first tine width to a second tine width, whereby the second tine width is smaller than the first tine width. The straight portion may comprise the second tine width. The second curved portion may comprise a tine width widening from the second tine width to the first tine width.

According to an embodiment, the anchoring arrangement may further comprise, in the relaxed configuration, an overall height of less than 5 mm. Alternatively or additionally, the anchoring arrangement may further comprise, in the relaxed configuration, an overall width of less than 15 mm. Alternatively or additionally, the anchoring arrangement may further comprise, in the relaxed configuration, an axial clearance distance between the cantilever end of the tines and the center axis of at least 4 mm.

The overall height of the tine may be a maximum dimension of the tine measured in a direction parallel to the central axis of the anchoring arrangement. In other words, such overall height extends from a most-distal portion of the tine to a most-proximal portion of the tine. Preferably, such overall height may be less than 4.8 mm, less than 4.6 mm, less than 4.4 mm or equal to or less than 4.3 mm.

The overall width of the tine may be a maximum dimension of the tine measured in a direction orthogonal to the central axis of the anchoring arrangement. In other words, such overall width extends from a left-most portion of the tine to a rightmost portion of the tine. Preferably, such overall width may be less than 14.8 mm, less than 14.6 mm or equal to or less than 14.4 mm.

The axial clearance distance may be a distance of the cantilever end of a tine with respect to the central axis of the anchoring arrangement. Such axial clearance distance should be sufficiently large such as to enable advantageous deformation characteristics during the deployment process and/or to avoid negative interactions with other components such as the protector sheath of the catheter and/or the housing of the IMD. In other words, setting the axial clearance distance to a sufficiently large value may increase a probability of engaging with the pectinate tissue at a cardiac wall during a deployment process while not interfering with an insertion tooling or an ILP housing. According to an embodiment, the anchoring arrangement comprises a first set of at least two longer tines and a second set of at least two shorter tines.

In other words, the anchoring arrangement may comprise at least two different types of tines, the types of tines differing from each other at least with respect to their length. For example, the length of the longer tines may be at least 10%, at least 20%, at least 30%, at least 40%, at least 50%, at least 60% or at least 70% longer than the length of the shorter tines.

The first set of tines may be configured for implementing other deformation characteristics than the second set of tines. For example, the first set of tines may be optimised for engaging with a first type of tissue whereas the second set of tines may be optimised for engaging with a different second type of tissue. For example, the first set of tines may be optimised for engaging with a tissue having protrusions such as pectinate structures whereas the second set of tines may be optimised for engaging with tissue having a smoother surface.

According to an embodiment, the longer tines and the shorter tines extend alternately from the fixation member.

In other words, every longer tine has a shorter tine as a direct neighbor along the circumference of the fixation member, and vice versa. The longer tines and/or the shorter tines may be arranged equidistantly with respect to each other. Such alternate arrangement of different tines may improve engaging characteristics of the anchoring arrangement upon being deployed at an implantation site.

According to an embodiment, each of the longer tines comprises, in the relaxed configuration, a first curved portion close to the fixation member and a second curved portion further distant from the fixation member. Furthermore, each of the shorter tines comprises, in the relaxed configuration, a single curved portion close to the fixation member and a straight portion further distant from the fixation member.

In the longer tines, the first curved portion may be separated from the second curved portion by an intermediate straight portion. Additionally or alternatively, the longer tines may comprise a straight portion in between the second curved portion and the distal cantilever end.

The shorter tines may differ from the longer tines with respect to their geometry in that they comprise only a single curved portion. Such single curved portion may be arranged close to the fixation member whereas a straight portion comprised in the shorter tines may follow the curved portion at a position further distant from the fixation member.

The different geometries of the longer tines and the shorter tines may be optimised for different deformation characteristics during a deployment process.

For example, according to an embodiment, the curved geometry and deformation characteristics of the longer tines and the shorter tines may be configured such as to implement the tines with the longer tines having a smaller emergence attack angle than the shorter tines, and/or with the longer tines having a larger maximum penetration depth than the shorter tines.

In other words, the first set of tines may be configured such as to implement a shallower deployment trajectory with a shallow penetration angle and/or a small maximum penetration depth. The second set of tines may then be configured such as to implement a steeper and/or more curved deployment trajectory with a steeper initial penetration angle and/or same or even smaller maximum penetration depth than is the case for the first set of tines.

In such configuration, upon deployment, the longer tines will engage first with the tissue. These tines may have a high probability of engaging with a region of the tissue having an uneven surface such as a pectinate muscle with a high ridge density. If the tissue region of deployment is smoother, the shorter set of tines that extend later during the deployment process may engage with the smooth tissue surface to ensure fixation. These shorter tines may have a steeper penetration angle but a reduced maximum penetration depth of for example less than 0.7 mm, less than 0.6 mm, less than 0.5 mm, less than 0.4 mm or equal or less than 0.3 mm. Particularly, the maximum penetration depth of the shorter tines may be less than the typical thickness of a thin atrial wall of an atrium chamber, therefore reducing any perforation risk.

It shall be noted that possible features and advantages of embodiments of the invention are described herein with respect to various embodiments of an anchoring arrangement or embodiments of an IMD comprising such anchoring arrangement. One skilled in the art will recognize that the features may be suitably transferred from one embodiment to another and features may be modified, adapted, combined and/or replaced, etc. in order to come to further embodiments of the invention.

Furthermore, it is to be noted that, while embodiments of the anchoring arrangement are described herein mainly with respect to an anchoring arrangement for an implantable leadless pacemaker, in particular an ILP configured for implantation within the atrium of a patient's heart, the anchoring arrangement may also be configured and used for anchoring other types of implantable medical devices.

In the following, advantageous embodiments of the invention will be described with reference to the enclosed drawings. However, neither the drawings nor the description shall be interpreted as limiting the invention.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

FIG. 1 shows a side view of an anchoring arrangement.

FIG. 2 shows a top view of the anchoring arrangement of FIG. 1.

FIG. 3 shows a side view of an implantable medical device.

FIGS. 4A, B show cross-sectional views of an implantable medical device during an initial phase of a deployment process and upon being finally fully deployed, respectively.

FIGS. 5A, B, 6A, B, 7A, B show side views of anchoring arrangements having second curved sections with differing sweep angles and cross-sectional views of the anchoring arrangement during initial phases of deployment processes, respectively.

FIGS. 8 to 11 show alternative anchoring arrangements.

FIG. 12 shows a side view of an anchoring arrangement having long tines and short tines.

FIG. 13 shows a side view of the anchoring arrangement of FIG. 12 at a different viewing angle.

FIG. 14 shows a perspective view of the anchoring arrangement of FIG. 12.

FIG. 15 shows a cross-sectional view of the anchoring arrangement of FIG. 12 at a medium phase during a deployment process.

The figures are only schematic and not to scale. Same reference signs refer to same or similar features.

### DETAILED DESCRIPTION

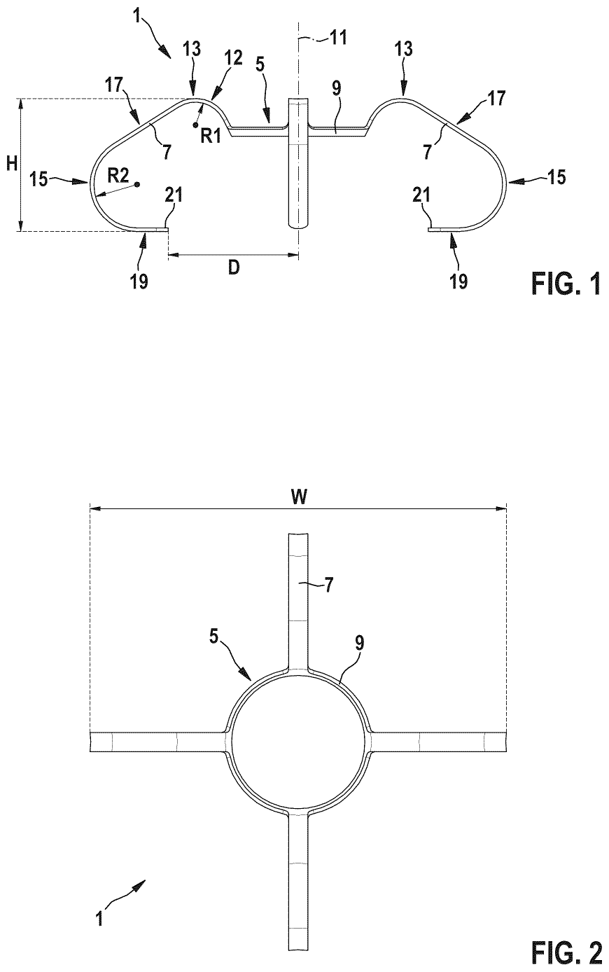

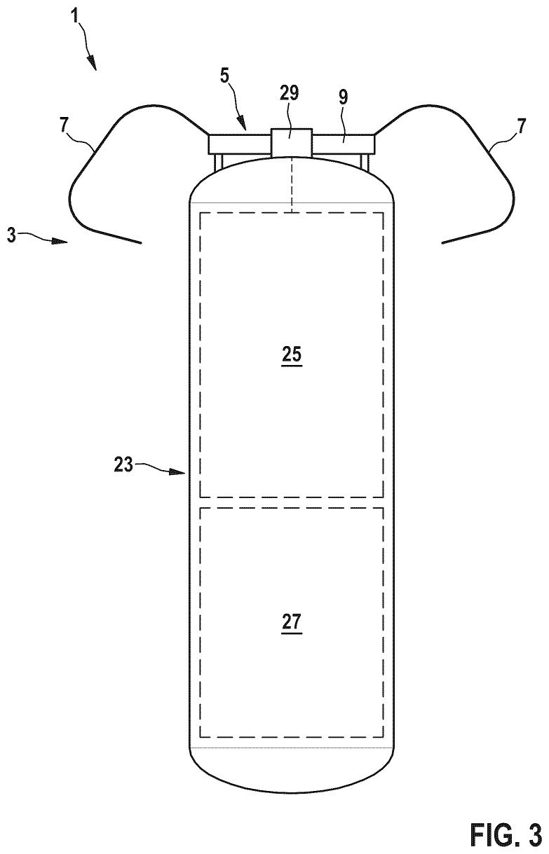

FIGS. 1 and 2 show an anchoring arrangement 1 for anchoring an implantable medical device at tissue of a patient, in particular for anchoring an ILP within the atrium of a heart. FIG. 3 shows an ILP 3 having such anchoring arrangement 1 fixed thereto.

The anchoring arrangement 1 comprises a fixation member 5 and four elongate tines 7 extending radially outwardly from the fixation member 5. The fixation member 5 is implemented as a circular ring 9. The tines 7 extend integrally from the ring 9 at opposite sides relative to a central axis 11 of the anchoring arrangement 1 and of the ring 9. The tines 7 extend at equidistant positions along a circumference of the ring 9.

FIG. 1 shows the anchoring arrangement 1 in a relaxed configuration in which the tines 7 are substantially free of any intrinsic mechanical prestress. In such relaxed configuration, each tine 7 comprises a middle portion 12 which is arranged distally relative to the fixation member 5 (close to the fixation member 5). Furthermore, a cantilever end 21 of each tine 7 is directed to an opposite proximal direction and/or is arranged proximally relative to the fixation member 5 (away from the fixation member 5).

Each of the tines 7 comprises a first curved portion 13 and a second curved portion 15. The first curved portion 13 is arranged close to the fixation member 5 whereas the second curved portion 15 is arranged further distant from the fixation member 5. Both curved portions 13, 15 are curved in a same direction. However, a radius of curvature R1 of the first curved portion 13 may differ from a radius of curvature R2 of the second curved portion 15. Furthermore, a proximal straight portion 17 extends between the first curved portion 13 and the second curved portion and a distal straight portion 19 extends between the second curved portion 15 and a cantilever end 21 at a free end of each of the tines 7. The tines 7 extend along an overall height H of 4.3 mm. An overall width W of the anchoring arrangement 1 is 14.4 mm, wherein such overall width may also be referred to as a diameter of the anchoring arrangement 1. An axial clearance distance D between each of the cantilever ends 21 of the tines 7, on the one side, and the central axis 11, on the other side, is 4.5 mm.

The tine array of the anchoring arrangement 1 is optimized with regards to characteristics of atrial tissue such as its thickness and elasticity. It could be made of a superelastic material, such as Nitinol, fabricated by laser cutting from a nitinol tube and setting to the desired shape. The tine leg typically has a consistent thickness and width along the entire length of the tine, and the tine tip may end in a radius along its width. The entire component would be subjected to finishing processes such as bead blasting, etching, and electropolishing in order to remove sharp edges and reduce a propensity for corrosion.

FIG. 3 shows an IMD 3 implemented as implantable leadless pacemaker, in particular configured for implantation within the atrium of a heart. The IMD 3 comprises a housing 23. A controller 25 is accommodated within the housing 23 and is configured for controlling functions of the IMD 3. An energy source 27 such as a battery is also accommodated within the housing 23 and is configured for supplying the controller 25 with electric energy. At a distal end of the IMD 3, an electrode 29 is exposed, i.e. the electrode 29 protrudes beyond the housing 23. The electrode 29 is electrically connected to the controller 25 such that electric voltage pulses generated by the controller 25 may be applied to the electrode 29. Furthermore, the IMD 3 comprises the anchoring arrangement 1 attached to the housing 23 at the distal end of the housing 23.

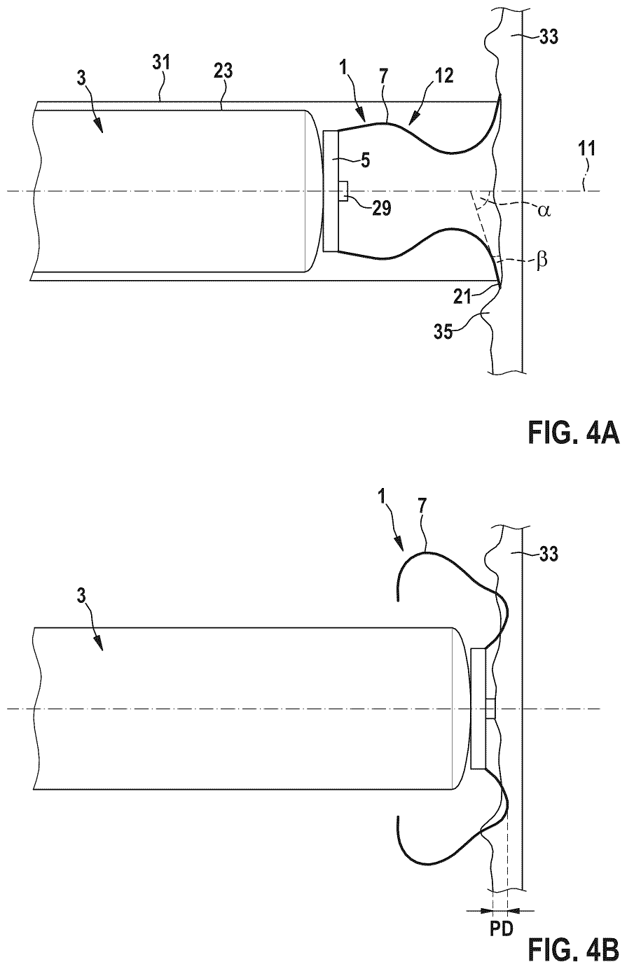

FIGS. 4A and 4B visualise different phases or stages during a deployment process for anchoring the IMD 3 to cardiac tissue 33 of e.g. an atrial wall. Prior to such a deployment process, a catheter having a protector sheath 31 at its distal end has been introduced into the patient's heart such that the distal end of the protector sheath 31 abuts against a surface of the cardiac tissue 33. Before starting the deployment process, the IMD 3 together with its anchoring arrangement 1 is completely accommodated within the protector sheath 31. Therein, the anchoring arrangement 1 is arranged distally in relation to the IMD 3 and the tines 7 are arranged in a pre-deployment configuration in which the tines 7 are mechanically deformed and intrinsically prestressed into a geometry such that the anchoring arrangement 1 may be included in the tubular protector sheath 31 with the middle portions 12 as well as the cantilever ends of the tines 7 being arranged and being directed distally relative to the fixation member 5.

Then, upon starting the deployment process as visualised in FIG. 4A, the catheter together with the protector sheath 31 is successively retracted such that the IMD 3 together with the anchoring arrangement 1 is successively ejected from the protector sheath 31 at the distal open end of the protector sheath 31. As the cantilever ends 21 of the tines 7 form the distal-most portions of the anchoring arrangement 1 in its pre-deployment configuration, it is these cantilever ends 21 which are first ejected from the protector sheath 31. Therein, depending on the geometry of the entire anchoring arrangement 1 and particularly depending on the shape and curvatures of the tines 7, the tines 7 with their cantilever ends 21 are arranged and ejected in a predetermined ejection direction. Furthermore, due to the tines 7 being deformed and therefore being intrinsically prestressed, the cantilever ends 21 of the tines 7 tend to move along a deployment trajectory in order to relax their intrinsic prestress upon being ejected from the protector sheath 31.

As visualised in FIG. 4A, such deployment trajectory, at the initial phase of the deformation procedure in the deployment process, extends at an emergence attack angle α relative to the central axis 11 of the anchoring arrangement 1. Such emergence attack angle α corresponds to a complementary penetration angle β which defines an angle between the deployment trajectory, at the initial phase of the deformation, relative to a surface of the cardiac tissue 33, such surface generally being arranged orthogonal to the central axis 11.

As described herein, the shape of the tines 7 is specifically optimised such as to enable a pre-deployment configuration and an initial phase of the deformation procedure in a deployment process such that the emergence attack angle α is between 40° and 90°, preferably between 75° and 90°, such that the penetration angle β is smaller than 50°, preferably smaller than 15°. Due to such shallow penetration angle β, the cantilever ends 21 of the tines 7 may penetrate the abutting cardiac tissue 33 at a shallow angle and thereby follow a shallow deployment trajectory during the deployment process.

In other words, when deployed from the protector cup for implant, the specific geometry of the tines 7 would make the tip of the tines 7 approach the tissue 33 at an angle α nearly perpendicular to the axis 11 of the protector cup, enabling an extremely shallow penetration depth PD. The primary direction of force from these tines 7 is in the radial direction, driving the tines 7 laterally through tissue 33, increasing the probability to engage with e.g. pectinate structures 35 of the tissue 33 while minimizing the likelihood of perforating the atrial wall.

Summarized, as a result of the deployment trajectory being shallow, the maximum penetration depth PD may be small and a risk of damaging or even perforating the cardiac tissue 33 is low even in a case in which the cardiac tissue 33 is a portion of a thin atrial wall enclosing an atrium chamber.

FIG. 4B shows a final phase of the deployment process when the anchoring arrangement 1 has been fully deployed and substantially reaches its relaxed configuration. Therein, during the deployment process, the tines 7 have been moved through a portion of the cardiac tissue 33 in order to engage behind such portion of the cardiac tissue 33 and thereby anchor the anchoring arrangement 1 together with the IMD 3 in the cardiac tissue 33. A maximum thickness of the portion engaged behind by the tines 7 of the anchoring arrangement 1 generally corresponds to the maximum penetration depth PD.

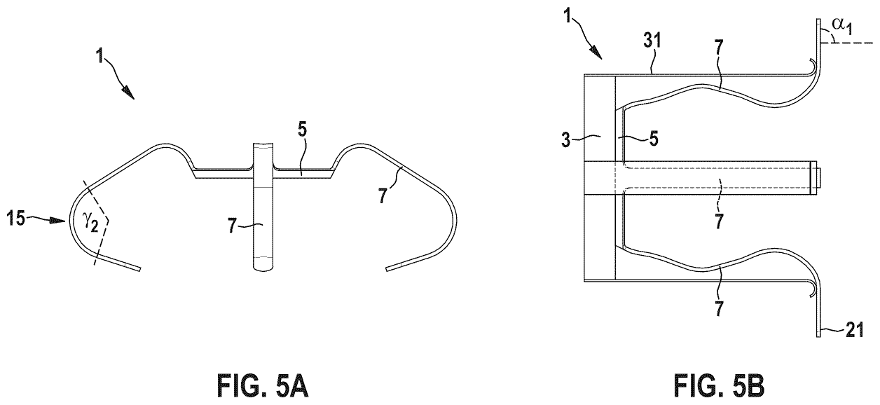

FIG. 5 to 7 show embodiments of the anchoring arrangement 1 which differ with regards to a sweep angle γ2 of the second curved portion 15. In FIG. 5, such sweep angle γ2 is 130°. In FIG. 6, the sweep angle γ2 is 115°. In FIG. 7, the sweep angle γ2 is 100°.

As apparent from FIGS. 5A, 6A, 7A, the size of the sweep angle γ2 directly influences the emergence attack angle α upon the anchoring arrangement 1 being ejected from the protector sheath 31 and being deployed from its pre-deployment configuration to its relaxed configuration. In the example shown, with the sweep angle γ2 of the second curved portion 15 being 130° (see FIG. 5B), the emergence attack angle α₁ is approximately 90°, whereas in the case of the sweep angle γ2 of the second curved portion 15 being 100° (see FIG. 7B), the emergence attack angle α₃ is approximately 60°.

In other words, the variations of embodiments shown in FIGS. 5-7 include incremental reductions in the sweep angle of the second radius, ranging from around 130° to around 100°. As the sweep angle decreases, the enclosed angle of the tine trajectory increases. This, in turn, increases both the angle of attack when loaded into a catheter and the estimated maximum penetration depth PD. The first embodiment visualized in FIG. 5 is designed for a lateral engagement only, with 0 mm of penetration depth by design. The embodiments visualized in FIGS. 6 and 7 increase the maximum allowed penetration depth from approximately 0.2 mm to 1 mm.

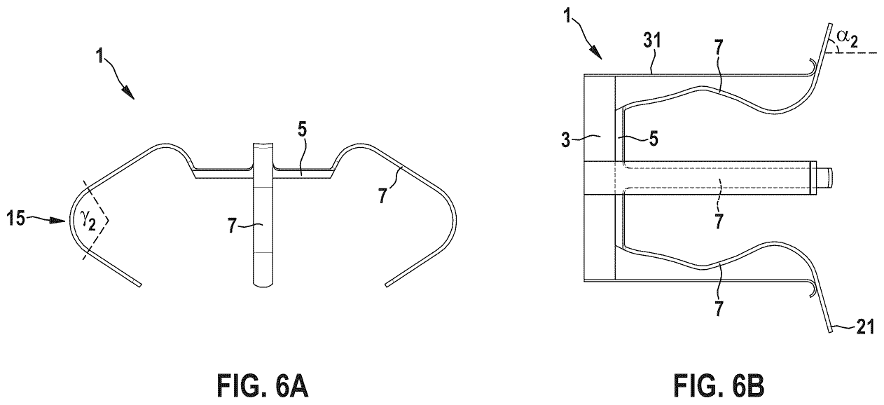

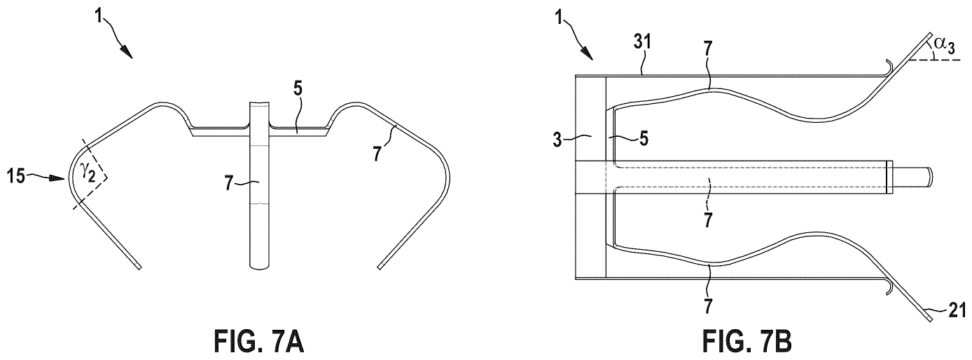

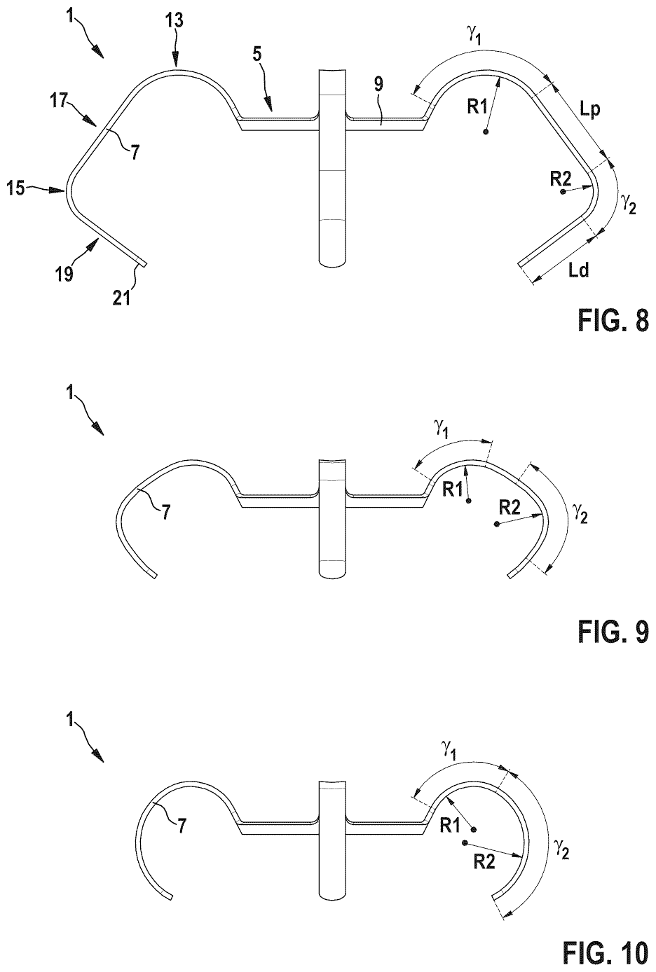

FIGS. 8 to 11 show various embodiments of the anchoring arrangement 1 with different geometries of the tines 7.

In the embodiment shown in FIG. 8, the first curved portion 13 includes a larger first radius R1 of approximately 1.5 mm with a sweep angle γ1 of 150°. The first curved portion 13 is followed by a proximal straight portion 17 having a length lp of approximately 2.54 mm. The second curved portion 15 includes a smaller second radius R2 of approximately 0.8 mm with a sweep angle γ2 of 90°. The second curved portion 15 is followed by a distal straight portion 19 having a length ld of approximately 2.22 mm. Such geometry may lower a strain in the first curved portion 13 when retracted into the implant tooling, and strengthen the spring of the second curved portion 15.

In the embodiment shown in FIG. 9, a tine profile geometry is downsized for optimising a fixation mechanism for the narrowing atrial appendage. In such embodiment, the straight segments (i.e. the proximal straight portion 17 and the distal straight portion 19) are removed and instead the tine profile is a series of continuously varying radii. The first prominent radius R1 is tangentially attached to the base ring 9 and is for example between 1 mm and 2 mm (1.02 mm is the example visualised in FIG. 9) with a sweep angle γ1 of approximately 90°. The spline extends the first radius to a second more prominent radius R2 of approximately 1.25 mm with a sweep angle γ2 of approximately 115°.

Alternatively, as shown in FIG. 10, the first radius R1 and second radius R2 could be increased slightly, such that the tine profile comprises of two distinct radii. The first radius, R1, would be tangentially attached to the base ring 9, would have a radius R1 of 1-2 mm and a sweep angle γ1 of approximately 90°. The second radius, R2 would be tangentially attached to R1, would have a radius R2 of 1-2 mm, and a sweep angle γ2 of approximately 120°.

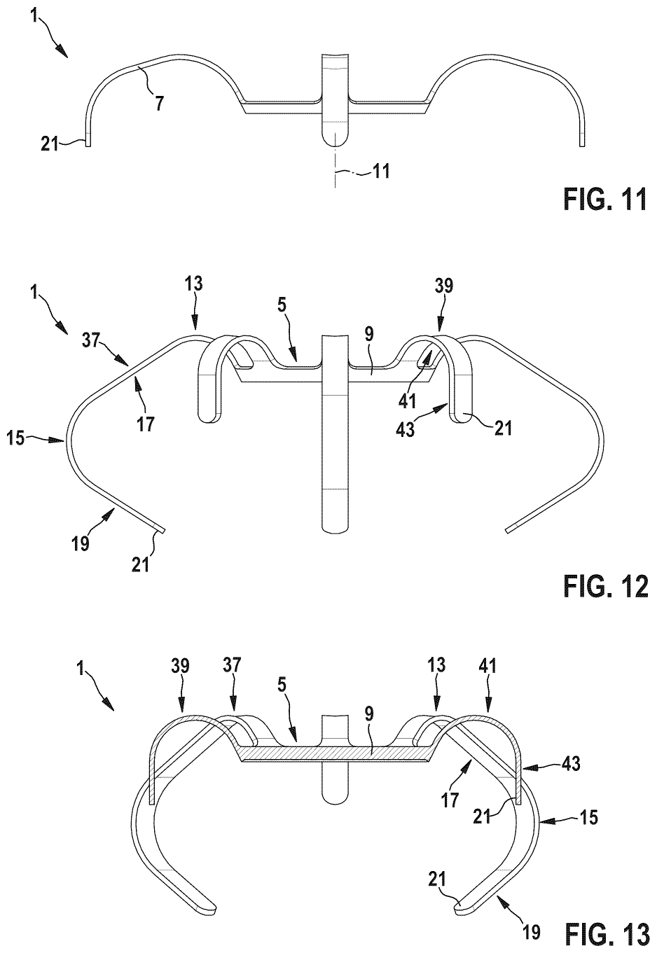

FIG. 11 shows another alternative embodiment of the anchoring arrangement 1. Therein, the tine profile is a spline-based geometry that ends with the tips at the cantilever ends 21 ending parallel to the central axis 11.

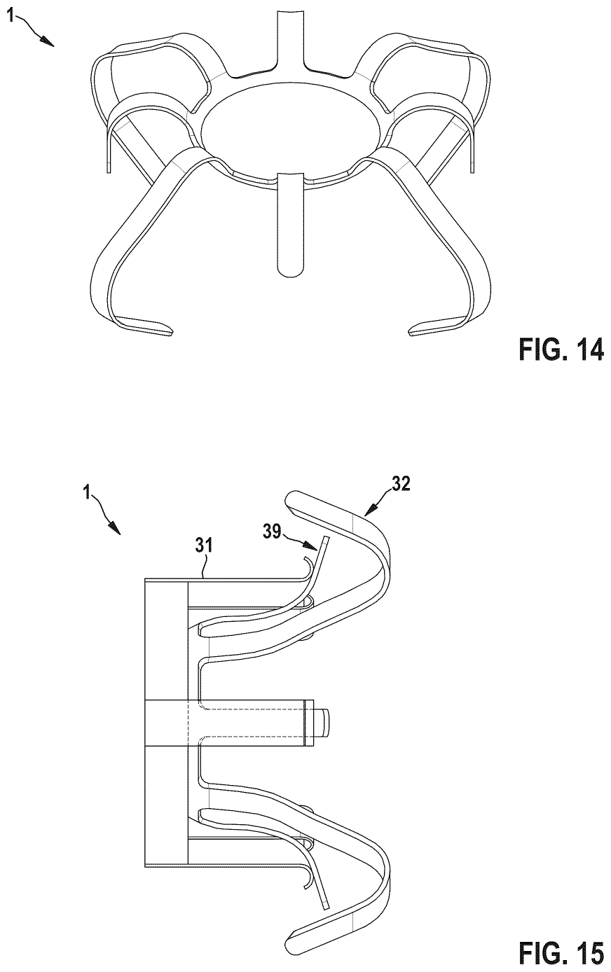

FIGS. 12 to 15 show an embodiment of an anchoring arrangement 1 having different types of tines 7. Specifically, the anchoring arrangement 1 comprises longer tines 37 as well as shorter tines 39. In the example shown, there are four longer tines 37 and four shorter tines 39 alternately extending from a circumference of the fixation member 5.

The longer tines 37, when in their relaxed configuration, have a profile similar or same to the profile discussed above with respect to the embodiments of FIGS. 1 and 5 to 11. Particularly, the longer tines 37 comprise a first curved portion 13, a second curved portion 15 and, optionally, proximal and distal straight portions 17, 19. The shorter tines 39 comprise only a single curved portion 41 close to the fixation member 5 and a straight portion 43 further distant from the fixation member 5.

Therein, the curved geometry and deformation characteristics of the longer tines 37 and of the shorter tines 39 are configured for implementing the tines such that the longer tines 37 have a smaller emergence attack angle than the shorter tines 39 and/or the longer tines 37 have a larger maximum penetration depth PD than the shorter tines 39, as visualised in the representation of an initial deployment stage shown in FIG. 15.

In other words, the embodiment shown in FIGS. 12 to 15 is a multi-tine array optimized for anatomic variation of the atrial wall including regions of pectinate tissue and smooth atrial wall. It could be made of a superelastic material, such as Nitinol, fabricated by laser cutting from a nitinol tube and setting to the desired shape. All tine legs are of consistent thickness and width along the entire length of the tine, and each tine tip would end in a radius. The entire component would be subjected to finishing processes such as bead blasting and electropolishing to remove sharp edges and reduce propensity for corrosion.

One set of tines 37 for this array, four depicted, is equidistant around the tine ring 9 and shape set as described above: starting at the transition from the base ring 9 linking all tines 37, 39 together, this set of longer tines 37 would consist of a small radius, followed by a long straight section, followed by a larger radius, followed by a short straight section. The alternating tines 39, four depicted, are shorter and consist of a radius followed by a straight section. In this embodiment, the first radii of all tines 37, 39 are all equivalent. The straight section of the shorter tines 39 are substantially equivalent to the central axis 11 of the device in the unrestrained state.

The longer tines 37 will engage first with the tissue upon deployment. These tines 37 will have a high probability of engaging within a region of pectinate muscle with high ridge density. If the region of deployment is smoother, then the set of shorter tines 39 that extend second will engage with the smooth atrial wall to ensure fixation. These tines have a higher angle α of attack, but a reduced maximum penetration depth PD of approximately 0.3 mm, which is less than the thickness of the thin atrial wall regions characterized in literature.

Finally, it should be noted that the term “comprising” does not exclude other elements or steps and the “a” or “an” does not exclude a plurality. Also elements described in association with different embodiments may be combined. It should also be noted that reference signs in the claims should not be construed as limiting the scope of the claims.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 anchoring arrangement
3 implantable medical device
5 fixation member
7 tine
9 ring
11 central axis
12 middle portion
13 first curved portion
15 second curved portion
17 proximal straight portion
19 distal straight portion
21 cantilever end
23 housing
25 controller
27 energy source
29 electrode
31 protector sheath
33 cardiac tissue
35 pectinate structure
37 longer tines
39 shorter tines
41 curved portion
43 straight portion
H overall height
W overall width
D axial clearance distance
A emergence attack angle
β penetration angle
PD maximum penetration depth
R1 radius of curvature of first curved portion
R2 radius of curvature of second curved portion
y1 sweep angle of first curved portion
γ2 sweep angle of second curved portion
lp length of proximal straight portion
ld length of distal straight portion

1. An anchoring arrangement configured for anchoring an implantable medical device, in particular an implantable leadless pacemaker, at tissue of a patient, the anchoring arrangement comprising
a fixation member for fixing the anchoring arrangement to the implantable medical device, and
at least two tines extending from the fixation member at different sides relative to a central axis of the anchoring arrangement, wherein the tines have a curved geometry and are elastically deformable such as to be reversibly deformable between a relaxed configuration and a pre-deployment configuration,
wherein, in the relaxed configuration, the tines are free of any intrinsic mechanical prestress and middle portions of the tines are arranged distally relative to the fixation member and cantilever ends of the tines are at least one of directed and arranged proximally relative to the fixation member, and
wherein, in the pre-deployment configuration, the tines are mechanically deformed and intrinsically prestressed into a geometry in which the anchoring arrangement is arrangeable in a tubular protector sheath with the middle portions as well as the cantilever ends of the tines being arranged and directed distally relative to the fixation member,
wherein the curved geometry and deformation characteristics of the tines are configured such that, in an initial phase of a deformation procedure during which the tines deform from their pre-deployment configuration to their relaxed configuration due to their intrinsic prestress, the cantilever ends of the tines are displaced such as to follow a deployment trajectory which extends at an emergence attack angle of between 40° and 90° relative to the central axis of the anchoring arrangement.

2. The anchoring arrangement of claim 1, wherein the curved geometry and deformation characteristics of the tines are configured such as to implement an emergence attack angle of between 75° and 90°.

3. The anchoring arrangement of claim 1, wherein the curved geometry and deformation characteristics of the tines are configured such that a maximum penetration depth of the tines is less than 1 mm, the maximum penetration depth corresponding to a maximum distance, in a direction parallel to the central axis of the anchoring arrangement, of the deployment trajectory in relation to a position of the cantilever ends of the tines upon the tines being in their pre-deployed configuration.

4. The anchoring arrangement of claim 1, wherein the curved geometry and deformation characteristics of the tines are configured such that an active maximum diameter of the anchoring arrangement is less than 15 mm, the active maximum diameter corresponding to a maximum width, in a direction orthogonal to the central axis of the anchoring arrangement, of the deployment trajectory.

5. The anchoring arrangement of claim 1, wherein each tine comprises, in the relaxed configuration, a first curved portion close to the fixation member and a second curved portion further distant from the fixation member.

6. The anchoring arrangement of claim 5, wherein the first curved portion has a first radius of curvature and the second curved portion has a second radius of curvature different from the first radius of curvature.

7. The anchoring arrangement of claim 5, wherein the second curved portion encloses a sweep angle of between 80° and 150°.

8. The anchoring arrangement of claim 5, wherein each tine further comprises, in the relaxed configuration, at least one of
a proximal straight portion in between the first curved portion and the second curved portion, and
a distal straight portion in between the second curved portion and the cantilever end.

9. The anchoring arrangement of claim 8, wherein the distal straight portion is oriented in parallel to the central axis of the anchoring arrangement.

10. The anchoring arrangement of claim 1, wherein the anchoring arrangement further comprises, in the relaxed configuration, at least one of
an overall height of less than 5 mm,
an overall width of less than 15 mm, and
an axial clearance distance between the cantilever end of the tines and the central axis of at least 4 mm.

11. The anchoring arrangement of claim 1, wherein the anchoring arrangement comprises a first set of at least two longer tines and a second set of at least two shorter tines.

12. The anchoring arrangement of claim 11, wherein the longer tines and the shorter tines extend alternately from the fixation member.

13. The anchoring arrangement of claim 11,
wherein each of the longer tines comprises, in the relaxed configuration, a first curved portion close to the fixation member and a second curved portion (15) further distant from the fixation member, and
wherein each of the shorter tines comprises, in the relaxed configuration, a single curved portion close to the fixation member and a straight portion further distant from the fixation member.

14. The anchoring arrangement of claim 11,
wherein the curved geometry and deformation characteristics of the longer tines and the shorter tines are configured such as to implement the tines with at least one of
the longer tines having a smaller emergence attack angle than the shorter tines, and
the longer tines having a larger maximum penetration depth than the shorter tines.

15. Implantable medical device comprising:
a housing,
a controller comprised in the housing for controlling functions of the medical device,
an energy source comprised in the housing for supplying the controller, and
an anchoring arrangement according to claim 1 with its fixation member being fixed to the housing.
